(19) United States
(12) Patent Application Publication
(10) Pub. No.: US 2006/0276412 A1
(43) Pub. Date: Dec. 7, 2006

(54) METHODS AND COMPOSITIONS FOR MANAGING PSYCHOTIC DISORDERS

(76) Inventor: Gary Tollefson, Indianapolis, IN (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/420,385

(22) Filed: May 25, 2006

Related U.S. Application Data
(60) Provisional application No. 60/686,128, filed on May 31, 2005.

(51) Int. Cl. A61K 31/7008; A61K 31/551; A61K 31/519; A61K 31/496; A61K 31/425; A61K 31/19

(52) U.S. Cl. 514/23; 514/220; 514/259.41; 514/373; 514/557; 514/253.06

(57) ABSTRACT

Compositions for treating psychotic disorders comprise a first ingredient and a second ingredient, wherein the first ingredient comprises at least one antipsychotic agent selected from the group consisting of ziprasidone, olanzapine and risperidone and the second ingredient comprises at least one anticonvulsant selected from the group consisting of zonisamide and topiramate. Methods of treating psychotic disorders, symptoms associated with psychotic disorders, and side effects associated with antipsychotic agents, comprise administering a first ingredient and a second ingredient, wherein the first ingredient comprises at least one antipsychotic agent selected from the group consisting of ziprasidone, olanzapine and risperidone and the second ingredient comprises at least one anticonvulsant selected from the group consisting of zonisamide and topiramate. The second ingredient of the compositions and methods may further comprise an antidepressant. In various embodiments, the antipsychotic agent and the anticonvulsant act synergistically to alleviate symptoms and/or side effects associated with psychotic disorders and their treatment.

### CROSS REFERENCE TO RELATED APPLICATION

This application claims priority to U.S. Provisional Application Ser. No. 60/686,128, filed on May 31, 2005, which is hereby incorporated by reference in its entirety.

### BACKGROUND OF THE INVENTION

1. Field of the Invention

The present invention relates to improved pharmaceutical compositions and methods for the treatment of psychotic disorders.

2. Description of the Related Art

Psychosis refers to a clinical state characterized by delusions (false beliefs) and/or hallucinations (sensory misperceptions). The more common of these disorders recognized by the American Psychiatric Association's Diagnostic and Statistical manual of Mental Disorders (DSM-IVTR) include bipolar disorder and schizophrenia. Bipolar disorder, also known as manic-depressive illness, is manifested by recurrent episodes of mania/hypomania, depression, or a combination of both (mixed episode). Each of these stages may manifest in psychosis or give rise to a risk for the emergence of psychosis. Schizophrenia is comprised of psychotic manifestations, often depressive elements, and disruption of the basic elements of an individual's personality structure. This syndrome typically lasts over a more protracted period of time than the classic cyclic nature (recurrence) of bipolar disorder. Other psychotic disorders include: borderline personality, delusional disorder, brief reactive psychosis, schizoaffective disorder, schizophreniform disorder, psychotic major depression, psychosis due to substance abuse, and psychoses associated with medical conditions e.g., dementia, delirium, etc.

Whereas many new treatment choices have emerged in the past decade for management of psychiatric disorders, their treatment remains an extremely difficult task for the clinician. Classical antipsychotic agents (e.g., haloperidol) are moderately effective but fail to alleviate many of the associated symptoms, such as mood changes. In fact, such agents can increase a patient's level of depression. Newer “atypical” antipsychotics can be slightly more effective (in schizophrenia or acute mania) but still fail to achieve a full remission (elimination of serious signs and symptoms) within the majority of patients treated. Moreover these agents fail to treat core disturbances in depressed mood. In contrast, while antidepressants treat downturns in mood (such as major depression) they must be used with great caution because of their potential for switching a bipolar patient's mood from depression to mania, or inducing a pattern of rapid-cycling mania/hypomania and depression. Within schizophrenia, antidepressants fail to treat the most prominent aspects of the illness. In summary, antidepressant drugs are not effective for psychotic symptoms when used alone. Given these limitations, clinicians have sometimes found it necessary to try mood-stabilizers such as lithium, valproate or carbamazepine. Olanzapine has also been a popular choice, as it is indicated for schizophrenia, acute mania, and bipolar maintenance. However it is not approved for either general psychosis or depression. Lastly, many of the above referenced drugs have safety concerns when used chronically. One such example is weight gain, which can increase considerably during treatment. Perhaps reflecting the scope of unmet medical need in such patient groups, market research reveals that typical patients receive three to four drugs at any one time. Thus, there is a growing need for identification of better treatment options that bestow a synergistic efficacy across the spectrum of both psychotic and mood symptoms while carrying less risk of long-term side effects such as weight gain.

Zonisamide is a novel anticonvulsant first developed in Japan. It is structurally similar to serotonin, a central indoleamine neurotransmitter that has been implicated in a number of psychiatric conditions, including psychosis and mood. Moreover, it possesses some pharmacologic actions, such as sodium and calcium channel antagonism. Zonisamide has a pharmacological profile that is very similar to that of several mood stabilizers. Thus, the effect of zonisamide was examined in 24 “psychotic” patients: 15 with bipolar manic state, 6 with schizoaffective manic state, and 3 schizophrenic excitement by Kanba and colleagues (1994). Approximately 25% of all the patients and 33% of the bipolar manic patients showed remarkable global improvement with the addition of zonisamide. Approximately 71% of all the patients and 80% of the bipolar group had more than moderate global improvement. More recently zonisamide has been reported to be a useful adjunctive treatment for some patients with bipolar depression (Baldassono et al, 2004).

Gadde et al have reported that zonisamide is associated with weight loss in obese individuals (Gadde et al, JAMA, 2003). McElroy and colleagues (2005) recently reported that, while data demonstrating an overlap between mood disorders and obesity can be coincidental, it appears to alternatively suggest that the two conditions are related. Of note, they found that adjunctive zonisamide was associated with beneficial effects on mood and body weight in some patients with bipolar disorders, but was also associated with a high discontinuation rate due to worsening mood symptoms. U.S. Patent Publication No. 2005/0181070 A1 discloses compositions of an anticonvulsant and a psychotherapeutic agent for the prevention of weight gain. U.S. Patent Publication No. 2005/0181070 A1 also discloses the simultaneous administration of olanzapine, zonisamide, valproate and bupropion to a patient, and the simultaneous administration of risperidone, zonisamide and paroxetine to a different patient.

U.S. Pat. No. 6,323,235 discloses the use of sulfamate derivatives such as topiramate for the treatment of impulse control disorders. U.S. Patent Publication No. 2005/0181070 A1 discloses a combination of (i) a first therapeutic agent which is an atypical antipsychotic and (ii) a second therapeutic agent selected from the group consisting of GABA modulators, anticonvulsants, and benzodiazepines, for use in treating a treatment-resistant anxiety disorder, a psychotic disorder or condition, or a mood disorder in a mammal. U.S. Patent Publication No. 2004/0002462 A1 discloses combination therapy for effecting weight loss that involves treating the subject with a combination of a sympathomimetic agent and an anticonvulsant sulfamate derivative.

There is a need for novel combination therapies that efficaciously treat the symptoms associated with psychotic disorders while avoiding undesirable side effects, such as weight gain.

### SUMMARY

Embodiments disclosed herein relate to pharmaceutical compositions and methods for treating psychotic disorders. In some embodiments, the pharmaceutical composition includes a first ingredient and a second ingredient, wherein the first ingredient includes an antipsychotic selected from ziprasidone, olanzapine, and risperidone, and wherein the second ingredient includes an anticonvulsant selected from zonisamide and topiramate. In some embodiments, the pharmaceutical composition does not include a combination of olanzapine, zonisamide, valproate and bupropion. In some embodiments, the pharmaceutical composition does not include a combination of risperidone, zonisamide and paroxetine.

In preferred embodiments, the antipsychotic can be ziprasidone and the anticonvulsant can be zonisamide. In other preferred embodiments, the antipsychotic can be ziprasidone and the anticonvulsant can be topiramate. In yet other preferred embodiments, the antipsychotic can be olanzapine and the anticonvulsant can be zonisamide. In still other preferred embodiments, the antipsychotic can be olanzapine and the anticonvulsant can be topiramate. In yet other preferred embodiments, the antipsychotic can be risperidone and the anticonvulsant can be zonisamide. In still other preferred embodiments, the antipsychotic can be risperidone and the anticonvulsant can be topiramate.

In some embodiments, the pharmaceutical composition also includes an antidepressant. For example, in preferred embodiments, the antidepressant can be a selective serotonin reuptake inhibitor. In other preferred embodiments, the antidepressant can be a tricyclic antidepressant. In still other preferred embodiments, the antidepressant can be a MAO inhibitor. In yet other preferred embodiments, the antidepressant can be a compound that enhances the activity of norepinephrine and/or dopamine.

Some embodiments relate to methods of treating a psychotic disorder including administering to a patient in need of treatment effective amounts of a first ingredient and a second ingredient, wherein the first ingredient includes at least one antipsychotic agent selected from ziprasidone, olanzapine, and risperidone, and the second ingredient includes at least one anticonvulsant selected from zonisamide and topiramate. In some embodiments, olanzapine, zonisamide, valproate and bupropion are not simultaneously administered to the patient. In some embodiments, risperidone, zonisamide and paroxetine are not simultaneously administered to the patient.

In preferred embodiments, the methods further include identifying a patient that is receiving ongoing treatment with an antipsychotic selected from ziprasidone, olanzapine, and risperidone. In other preferred embodiments, the methods further include identifying a patient that is suffering from a psychotic disorder associated with one or more symptoms in need of treatment. In still other preferred embodiments, the methods include identifying a patient that is suffering from a psychotic disorder that is in need of mood stabilization.

In some embodiments of the methods described herein, the psychotic disorder is selected from bipolar disorders, schizophrenia, borderline personality, schizoid/schizotypical/paranoid personality disorders, delusional disorder, belief reactive psychosis, schizoaffective disorder, schizophreniform disorder, psychotic major depression, psychosis due to substance abuse, psychosis associated with disorders of development, and a psychosis associated with medical conditions. For example, in some embodiments, the psychosis associated with medical conditions can be dementia, delirium, mental retardation, and the like.

These and other embodiments are described in greater detail below.

### DETAILED DESCRIPTION OF THE PREFERRED EMBODIMENTS

When used herein, the following terms and their grammatical equivalents have the definitions given below, in addition to their ordinary and customary meanings.

The term “treating” or its grammatical equivalents does not necessarily mean total cure. Any alleviation of any undesired signs or symptoms of the disease to any extent or the slowing down of the progress of the disease can be considered treatment. Furthermore, treatment can include acts that can worsen the patient's overall feeling of well being or appearance. Treatment can also include lengthening the life of the patient, even if the symptoms are not alleviated, the disease conditions are not ameliorated, or the patient's overall feeling of well being is not improved.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound disclosed herein with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound disclosed herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts thereof with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with the formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide can be an amino acid or a peptide molecule attached to a molecule disclosed herein, thereby forming a prodrug.

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

The term “metabolite” refers to a compound to which an active compound of the embodiments disclosed herein is converted within the cells of a mammal. The pharmaceutical compositions disclosed herein can include one or more metabolites of the compounds described herein. The scope of the methods of the embodiments disclosed herein includes those instances where a compound disclosed herein is administered to the patient, yet the metabolite of the compound is the bioactive entity.

A “prodrug” refers to an agent that is converted into the parent drug in vivo. Prodrugs are often useful because, in some situations, they can be easier to administer than the parent drug. They can, for instance, be bioavailable by oral administration whereas the parent is not. The prodrug can also have improved solubility in pharmaceutical compositions over the parent drug, or can demonstrate increased palatability or be easier to formulate. An example, without limitation, of a prodrug would be a compound disclosed herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

Throughout the present disclosure, when a particular compound is mentioned by name, for example, bupropion, it is understood that the scope of the present disclosure encompasses pharmaceutically acceptable salts, esters, amides, metabolites, or prodrugs of the named compound. Also, if the named compound comprises a chiral center, the scope of the present disclosure also includes compositions comprising the racemic mixture of the two enantiomers, as well as compositions comprising each enantiomer individually substantially free of the other enantiomer. Thus, for example, contemplated herein is a composition comprising the S enantiomer substantially free of the R enantiomer, or a composition comprising the R enantiomer substantially free of the S enantiomer. By “substantially free” it is meant that the composition comprises less than 10%, or less than 8%, or less than 5%, or less than 3%, or less than 1% of the minor enantiomer. If the named compound comprises more than one chiral center, the scope of the present disclosure also includes compositions comprising a mixture of the various diastereomers, as well as compositions comprising each diastereomer substantially free of the other diastereomers. Thus, for example, commercially available bupropion is a racemic mixture comprising two separate enantiomers. The recitation of “bupropion” throughout this disclosure includes compositions that comprise the racemic mixture of bupropion, the compositions that comprise the (+) enantiomer substantially free of the (−) enantiomer, and the compositions that comprise the (−) enantiomer substantially free of the (+) enantiomer.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

In one aspect, provided herein are compositions for the treatment of a psychotic disorder comprising a first ingredient and a second ingredient, wherein the first ingredient comprises at least one antipsychotic agent, and the second ingredient comprises at least one anticonvulsant. In various embodiments, the combination of the first ingredient and the second ingredient can have an enhanced efficacy in the treatment of a psychotic disorder and/or one or more symptoms associated with a psychotic disorder. In some embodiments, the first ingredient can exert a synergistic effect with the second ingredient with regard to the treatment of a psychotic disorder and/or one or more symptoms associated with a psychotic disorder.

In some aspects, the compositions disclosed herein can improve patient compliance in self-administering medications for the treatment of psychotic disorders. In additional embodiments, the compositions disclosed herein can have a mood stabilizing effect.

In some embodiments, the antipsychotic agent is a “typical antipsychotic.” Examples of typical antipsychotics include, but are not limited to, chlorpromazine, fluphenazine, haloperidol, molindone, thiothixene, thioridazine, trifluoperazine, perphenazine, and loxapine.

In other embodiments, the antipsychotic agent is an “atypical antipsychotic.” Atypical antipsychotics are a newer generation of antipsychotic drugs less likely to be associated with neurological adverse effects such as parkinsonian symptoms, tardive dyskinesia, and akathesia, as compared with traditional antipsychotics. Atypical antipsychotics are thus preferred for use in the embodiments disclosed herein. Currently marketed atypical antipsychotics include, but are not limited to, olanzapine (e.g., Zyprexa®), risperidone (e.g., Risperdal®), quetiapine (e.g., Seroquel®), ziprasidone (e.g., Geodon®), aripiprazole (e.g., Abilify®), and sertindole (e.g., Serdolect®), with olanzapine and risperidone being particularly preferred. Clozapine (e.g., Clozaril®) is also regarded as an atypical antipsychotic, however, it is not a first-line treatment because of it is associated with a high incidence of agranulocytosis.

In preferred embodiments, the antipsychotic agent is ziprasidone. Ziprasidone has the following chemical structure:

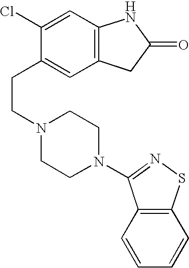

Ziprasidone has a high affinity for dopamine, serotonin, and alpha-adrenergic receptors and a medium affinity for histaminic receptors. Ziprasidone also displays some inhibition of synaptic reuptake of serotonin and norepinephrine. Without wishing to be bound by any particular theory, it is believed that the antipsychotic activity of ziprasidone is mediated primarily by antagonism at dopamine receptors (specifically the dopamine D₂ receptor), as well as its activity as a serotonin antagonist.

In other preferred embodiments, the antipsychotic is olanzapine.

Olanzapine has the following chemical structure:

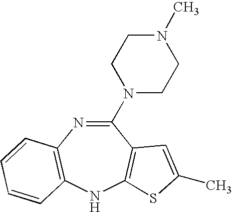

Olanzapine is classified as a thienobenzodiazepine. Olanzapine has a high affinity for dopamine and serotonin receptors and a lower affinity for histamine, cholinergic muscarinic and alpha adrenergic receptors. Without wishing to be bound by any particular theory, it is believed that olanzapine's antipsychotic activity is mediated primarily by antagonism at dopamine receptors (specifically the dopamine D₂ receptor), and its activity as a serotonin antagonist.

In some embodiments, the first ingredient can also comprise an antibipolar drug, including but not limited to, lithium, valproic acid, valproate, divalproex, carbamezepine, oxycarbamezepine, lamotrogine, tiagabine, and benzodiazepines.

In some embodiments, the anticonvulsant with sodium channel blocking activity is a compound of structural Formula (I): 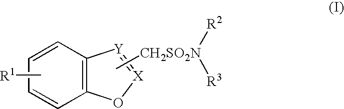
wherein R¹ is hydrogen or a halogen atom, R² and R³ are the same or different and are each hydrogen or an alkyl having 1 to 3 carbon atoms, and one of X and Y is a carbon atom and another is a nitrogen atom, provided that the group —CH₂SO₂NR²R³ is bonded to the carbon atom of either of X and Y, or an alkali metal salt thereof.

In some embodiments, the compound of structural Formula (I) is zonisamide. Zonisamide is a marketed anticonvulsant indicated as adjunctive therapy for adults with partial onset seizures. It is believed that the mechanism of antiepileptic activity is related to: 1) sodium-channel blocking; and/or, 2) reduction of inward T-type calcium currents. In addition to its antiepileptic activity, the present inventors have discovered that the combination of zonisamide with an antipsychotic medication is highly effective in the treatment of psychotic disorders and their associated symptoms. Without being bound by any particular theory, the psychotherapeutic effects of zonisamide can be related to the ability of zonisamide to facilitate serotonergic and dopaminergic neurotransmission. For example, there is evidence that zonisamide increases serotonin and dopamine synthesis rates (Hashiguti et al, J Neural Transm Gen Sect. 1993; 93:213-223; Okada et al, Epilepsy Res. 1992; 13:113-119, both of which are incorporated by reference herein in their entirety). There is also evidence suggesting that zonisamide stimulates dopamine D₂ receptors (Okada et al, Epilepsy Res. 1995; 22:193-205, incorporated by reference herein in its entirety). In addition, zonisamide binds to the GABA/benzodiazepine receptor complex without producing change in chloride flux, and has a weak inhibitory effect on carbonic anhydrase. With regard to the pharmacokinetics of zonisamide, its renal excretion and minimal potential for inhibition or induction of hepatic microsomal enzymes, are favorable qualities for combination use with antipsychotics. Zonisamide is well tolerated, with fatigue being the only side effect that occurs more frequently than with placebo treatment.

In some embodiments, the anticonvulsant with sodium channel blocking activity is a compound of structural Formula (II): 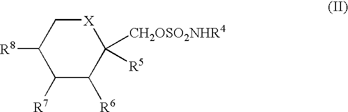
wherein X is CH₂ or oxygen, R⁴ is hydrogen or C₁₋₆ alkyl, R⁵, R⁶, R⁷ and R⁸ are independently hydrogen, C₁₋₄ alkyl or C₁₋₄ alkoxy, and when X is CH₂, R⁷ and R⁸ can be alkene groups joined to form a benzene ring, and when X is oxygen, R⁵ and R⁶ and/or R⁷ and R⁸ together can be a methylenedioxy group of the following Formula (III): 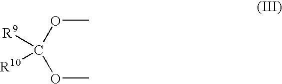
wherein R⁹ and R¹⁰ are the same or different and are each independently hydrogen, C₁₋₄ alkyl or C₆₋₁₀ aralkyl. R⁹ and R¹⁰ may be joined to form a cyclopentyl or cyclohexyl ring.

In some embodiments, the anticonvulsant of structural Formula II is topiramate. In addition to its antiepileptic/sodium channel blocking activity, the present inventors have discovered that the combination of topiramate with an antipsychotic medication is highly effective in the treatment of psychotic disorders and their associated symptoms.

In some embodiments, the anticonvulsant can be selected from the group consisting of the compounds of Formula (I), as described herein, zonisamide, the compounds of Formula (II), as described herein, topiramate, nembutal, lorazepam, clonazepam, clorazepate, tiagabine, gabapentin, fosphenyloin, phenyloin, carbamazepine, valproate, felbamate, levetiracetam, oxcarbazepine, lamotrigine, methsuximide, ethosuxmide, and other weight-loss promoting anticonvulsants (including agents that block kainate/AMPA (D,L-α-amino-3-hydroxy-5-methyl-isoxazole propionic acid) subtype glutamate receptors).

In additional embodiments, other methane-sulfonamide derivatives, in addition to zonisamide and topiramate, such as those described in U.S. Pat. No. 4,172,896, incorporated by reference herein in its entirety, or other sulfamates (including sulfamate-substituted monosaccharides), such as those described in U.S. Pat. No. 4,513,006, incorporated by reference herein in its entirety, are used as the weight loss promoting anticonvulsant.

The present inventors have discovered combinations of antipsychotics and anticonvulsant can synergistically enhance the efficacy of the antipsychotic agent. Patients treated with these combinations can show marked improvement in their psychotic symptoms to the extent not observed in the patients treated with the antipsychotic agent alone. The better results obtained by using the pharmaceutical compositions described herein encourages patients to continue with their therapies and thereby increasing patient compliance.

In some embodiments, the second ingredient enhances the efficacy of the compositions disclosed herein in treating psychotic disorders by alleviating one or more side effects associated with the administration of the antipsychotic agent(s) of the first ingredient, For example, the administration of many antipsychotic agents leads to significant weight gain as a side effect. The weight gain risk associated with many atypical antipsychotics is a major concern, particularly for patients that require chronic therapy (Allison et al, Am. J. Psych. 156:1686-1696 (1999)). Weight gain is reported as the most problematic side effect in patients treated with olanzapine, and this problem does not appear to be dose-related (Wirshing et al, J. Clin. Psych. 60:358-363 (1999)). In one study, the average weight gain at one year in olanzapine treated patients was 12 kg, and an analysis of four studies of various durations has shown a weight gain of ≧7% of 40% of olanzapine-treated patients compared with 12% in those receiving haloperidol and 3% in the placebo group (Weiden et al, J. Clin. Psych. 57:S53-S60 (1996), Beasley et al, J. Clin. Psych. 60:767-770 (1997)). The olanzapine-induced weight gain is noticeable in the first month of treatment, peaking at about 9 months. An increase in triglyceride levels has also been reported in olanzapine-treated patients (Osser et al, J. Clin. Psych. 60:767-770 (1998); Sheitman et al, Am. J. Psych. 156:1471-1472 (1999)). Weight gain has also been associated with treatment with risperidone and quetiapine. Of further concern is an observed increased prevalence of conditions associated with weight gain, such as type II diabetes, in patients treated with atypical antipsychotics (Ebenbichler et al, J. Clin. Psych. 64:1436-1439 (2003), Hedenmalm et al, Drug Saf. 25:1107-1116 (2002), Semyak et al, Am. J. Psych. 159:561-566 (2002)).

Thus, weight gain and other undesirable side effects associated with treatment with antipsychotic agents can occur in a large proportion of patients, be significant in magnitude, and be difficult to reverse, even after discontinuance of treatment. Such side effects can be a major reason for noncompliance with psychotherapy (see e.g., Cash et al, Percep. Motor Skills 90:453-456 (2000); Deshmukh et al, Cleveland Clinic J. Med. 70:614-618 (2003)).

In some embodiments, the anticonvulsant with sodium channel-blocking activity has a weight loss-promoting effect. In certain embodiments, the anticonvulsant with sodium channel-blocking activity is effective in promoting weight loss in a mammal. The mammal can be selected from the group consisting of mice, rats, rabbits, guinea pigs, dogs, cats, sheep, goats, cows, primates, such as monkeys, chimpanzees, and apes, and humans. In certain embodiments, the weight loss-promoting anticonvulsant with sodium channel-blocking activity alleviates weight gain associated with the administration of the antipsychotic agent of the pharmaceutical compositions described herein, leading to increased patient compliance with, for example, self-administering compositions disclosed herein. In additional embodiments, the weight loss-promoting anticonvulsant with sodium channel-blocking activity allows for more effective treatment of overweight or obese individuals suffering from a psychotic disorder (e.g., individuals having a body mass index (BMI) greater than 25, 30, 35, or 40).

In certain embodiments, the weight-loss promoting anticonvulsant with sodium channel-blocking activity is zonisamide. In addition to zonisamide's anticonvulsant and psychotherapeutic effects, described above, zonisamide has also been shown to cause significant weight loss (comparable to marketed weight loss medications) in patients presenting with primary obesity (Gadde et al, JAMA 289:1820-1825 (2003), incorporated by reference herein in its entirety). In certain other embodiments, the weight-loss promoting anticonvulsant is topiramate, which has also been shown to be effective as an anti-obesity agent. Advantageously, the administration of zonisamide or topiramate in combination with an antipsychotic medication prevents or decreases undesirable weight gain and/or additional side effects associated with the antipsychotic medication, increasing patient compliance with treatments that involve administering compositions disclosed herein. Preferably, olanzapine, zonisamide, valproate and bupropion are not simultaneously administered to the patient. Preferably, risperidone, zonisamide and paroxetine are not simultaneously administered to the patient.

In addition to causing various side effects, such as weight loss, currently available antipsychotic agents have limited efficacy in treating many psychological symptoms, such as mood disorders and depression. Thus, in some embodiments, either one or both of the first and second ingredients comprises an antidepressant. For example, in an embodiment, the second ingredient comprises a combination of an anticonvulsant with sodium channel-blocking activity and an antidepressant. Advantageously, the combination of the antidepressant with an anticonvulsant with sodium channel-blocking activity and an antipsychotic agent enhances the effectiveness of the compositions disclosed herein in treating psychotic disorders and their symptoms. In some embodiments, the combination of an antidepressant with an anticonvulsant with sodium channel-blocking activity and an antipsychotic agent alleviates mood disorders and/or depression in patients suffering from psychotic disorders. In further embodiments, the mood disorder and/or depression is part of the etiology of the psychotic disorder, while in other embodiments they comprise additional conditions in need of treatment. In yet additional aspects, the mood disorders and/or depression are side effects of the administration of one or more antipsychotic agents.

In some embodiments, the combination of an antidepressant with an anticonvulsant with sodium channel-blocking activity and an antipsychotic agent has a mood stabilizing effect on a patient suffering from a psychotic disorder. In some aspects, the mood stabilizing effect directly treats the symptoms of the psychotic disorder, while in some aspects the mood stabilizing effect indirectly enhances the efficacy of treatment by improving patient compliance.

Without being bound to a particular theory, the present inventors believe that the addition of an anticonvulsant with sodium channel-blocking activity to the antidepressant or antipsychotic therapy has certain physiological and biochemical advantages. For instance, the addition of an anticonvulsant, such as zonisamide or topiramate, to the antidepressant therapy has the effect of enhancing certain serotonergic activities associated with atypical antipsychotics. Further, the addition of the anticonvulsant mitigates the weight gain associated with 5-HT2C antagonism. In addition, combinations of an anticonvulsant with sodium channel-blocking activity with an antipsychotic introduces a synergistic effects via ionic channel regulation/intracellular events at second/third level intracellular messenger systems (ex. cAMP, cGMP, etc), in turn, influencing the expression of gene mediated protein synthesis/production of trophic factors, ionic flow into and out of the cell, and the like.

In some embodiments, antidepressants useful in the compositions can include, but are not limited to, selective serotonin reuptake inhibitors (e.g., fluoxetine, fluvoxamine, sertraline, paroxetine, citalopram, and escitalopram), tricyclic antidepressants (e.g., imipramine, desipramine, trimipramine, nortriptyline, clomipramine, doxepin, amitriptyline, maprotiline, protriptyline, dothiapen, and maprotiline), MAO inhibitors (e.g., phenelzine (e.g., Nardil®), tranylcypromine (e.g., Parnate®), isocarboxazid (e.g., Marplan®) and moclobemide (e.g., Aurorix®), norepinephrine reuptake inhibitors (e.g., atomoxetine, bupropion, thionisoxetine, and reboxetine), mixed dopamine/norepinephrine reuptake inhibitors (e.g., bupropion), nefazodone, mianserin setiptiline, viqualine trazodone, cianopramine, and mixed serotonin/norepinephrine uptake inhibitors duloxetine (e.g., Cymbalta®), venlafaxine (e.g., Effexor®), and/or mirtazapine. Additional antidepressants useful in the compositions disclosed herein are disclosed in U.S. Pat. Nos. 3,819,706 and 3,885,046, incorporated by reference herein in their entirety.

In a preferred aspect, the antidepressant comprising the second ingredient, in combination with the at least one anticonvulsant with sodium channel-blocking activity, is bupropion. Bupropion exerts its antidepressant effects via a dual mechanism of norepinephrine and dopamine reuptake inhibition. Bupropion has a unique pharmacological profile compared to other antidepressants currently on the market in that bupropion does not affect serotonin or directly, postsynaptic receptors. Bupropion's unique pharmacological properties allow it to be used in the treatment of depression and other mood disorders with minimal side effects, such as sexual dysfunction, weight gain, and sedation that are prevalent with the use of other commonly prescribed antidepressants. Moreover, the present inventors have shown that bupropion has synergistic effects with both zonisamide and topiramate in treating obesity. Thus, the combination of bupropion with the anticonvulsant with sodium channel-blocking activity and antipsychotic agents of the compositions described herein is particularly effective in the treatment of psychotic disorders in overweight or obese patients (e.g., having a BMI greater than 25). While the use of bupropion is also preferred, compounds disclosed in U.S. Pat. Nos. 3,819,706 and 3,885,046 can be used, as can other compounds that enhance the activity of norepinephrine and/or dopamine via uptake inhibition or other mechanism (e.g., Atomoxetine® or Reboxetine®).

In some embodiments, the compound that enhances the activity of norepinephrine and/or dopamine via uptake inhibition or other mechanism is a metabolite of bupropion. The metabolites of bupropion suitable for inclusion in the methods and compositions disclosed herein include the erythro- and threo-amino alcohols of bupropion, the erythro-amino diol of bupropion, and morpholinol metabolites of bupropion. In some embodiments, the metabolite of bupropion is (±)-(2R*,3R*)-2-(3-chlorophenyl)-3,5,5-trimethyl-2-morpholinol. In some embodiments the metabolite is (−)-(2R*,3R*)-2-(3-chlorophenyl)-3,5,5-trimethyl-2-morpholinol, while in other embodiments, the metabolite is (+)-(2S,3S)-2-(3-chlorophenyl)-3,5,5-trimethyl-2-morpholinol. Preferably, the metabolite of bupropion is (+)-(2S,3S)-2-(3-chlorophenyl)-3,5,5-trimethyl-2-morpholinol, which is known by its common name of radafaxine.

In some embodiments, the metabolite of bupropion is (+)-(2S,3S)-2-(3-chlorophenyl)-3,5,5-trimethyl-2-morpholinol hydrochloride. This metabolite is described in U.S. Pat. No. 6,274,579, issued on Aug. 14, 2001 to Morgan et al., which is hereby incorporated by reference herein in its entirety, including any drawings.

In another aspect, provided herein are pharmaceutical compositions wherein the compositions disclosed herein further comprise a physiologically acceptable carrier, diluent, or excipient, or a combination thereof. In some embodiments, the first ingredient and/or the second ingredient comprise two or more compounds joined together by a chemical linkage, such as a covalent bond, so that the two or more compounds comprising the first and second ingredients form separate parts of the same molecule. The chemical linkage is preferably selected such that after entry into the body, the linkage is broken, such as by enzymatic action, acid hydrolysis, base hydrolysis, or the like, and the two separate compounds are then formed.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application can be found in “Remington's Pharmaceutical Sciences,” Mack Publishing Co., Easton, Pa., 18th edition, 1990. In some embodiments, the pharmaceutical compositions do not include a combination of olanzapine, zonisamide, valproate and bupropion. In other embodiments, the pharmaceutical compositions do not include a combination of risperidone, zonisamide and paroxetine.

Suitable routes of administration can, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one can administer the compound in a local rather than systemic manner, for example, via injection of the compound directly in the renal or cardiac area, often in a depot or sustained release formulation. Furthermore, one can administer the drug in a targeted drug delivery system, for example, in a liposome coated with a tissue-specific antibody. The liposomes will be targeted to and taken up selectively by the organ.

The pharmaceutical compositions disclosed herein can be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the embodiments disclosed herein thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients can be used as suitable and as understood in the art; e.g., in Remington's Pharmaceutical Sciences, above.

For injection, the agents of the compositions disclosed herein can be formulated in aqueous solutions or lipid emulsions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacqucr solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Furthermore, the formulations of the embodiments disclosed herein can be coated with enteric polymers. All formulations for oral administration should be in dosages suitable for such administration.

For buccal administration, the compositions can take the form of tablets or lozenges formulated in conventional manner.

For administration by inhalation, the compounds for use in the embodiments disclosed herein can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein can be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of POLYSORBATE 80™; the fraction size of polyethylene glycol can be varied; other biocompatible polymers can replace polyethylene glycol, e.g., polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

Many of the compounds used in the pharmaceutical compositions disclosed herein can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the embodiments disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage can be a single one or a series of two or more given in the course of one or more days, as is needed by the patient. Note that for almost all of the specific compounds mentioned in the present disclosure, human dosages for treatment of at least some condition have been established. Thus, in most instances, the embodiments disclosed herein will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient can be, for example, an oral dose of between 0.1 mg and 500 mg, preferably between 1 mg and 250 mg, e.g. 5 to 200 mg or an intravenous, subcutaneous, or intramuscular dose of between 0.01 mg and 100 mg, preferably between 0.1 mg and 60 mg, e.g. 1 to 40 mg of the pharmaceutical compositions disclosed herein, or a pharmaceutically acceptable salt thereof calculated as the free base, the composition being administered 1 to 4 times per day. Alternatively the compositions disclosed herein can be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

For example, in some embodiments, the dosage range for zonisamide, for an oral dose, is in the range of about 25 to about 800 mg per day. Preferably the dose is from about 100 mg to 600 mg per day, more preferably from about 200 mg to 400 mg per day. In yet other embodiments, the dosage is 25 mg per day, 50 mg per day, or 100 mg per day. The daily dosage range for topiramate can be from about 25 mg to 1600 mg, preferably from about 50 mg to 600 mg, and more preferably from about 100 mg to 400 mg. The daily dosage range for bupropion can be from about 25 mg to 600 mg, preferably from about 50 mg or about 150 mg to 450 mg. The above doses generally are given once per day or divided (e.g., equally) into multiple doses. When zonisamide or topiramate are used in combination with bupropion, the ratio of zonisamide or topiramate to bupropion can range, for example, from about 2:1 to 1:2. The above ranges are given as non-limiting examples, and it can be necessary in some embodiments to use doses outside of the recited ranges.

In other examples, the daily dosage regimen of the antipsychotic agent risperidone for an adult human patient can be, for example, an oral dose of between 0.1 mg and 10 mg, preferably between 1 mg and 5 mg, of the pharmaceutical compositions disclosed herein, or a pharmaceutically acceptable salt thereof calculated as the free base, the composition being administered 1 to 4 times per day (e.g. in equally divided doses). Suitably, risperidone is administered for a period of continuous therapy, for example for several weeks or more, or for months or years. In yet another example, the daily dosage regimen of the antipsychotic olanzapine for an adult human patient can be, for example, an oral dose of between 1 mg and 100 mg, preferably between 2.5 mg and 50 mg, of the pharmaceutical compositions disclosed herein, or a pharmaceutically acceptable salt thereof calculated as the free base, the composition being administered 1 to 4 times per day (e.g. in equally divided doses). Olanzapine can administered in a dose of 2.5 mg, 5 mg, 10 mg, 15 mg, or 20 mg or higher. Suitably, olanzapine is administered for a period of continuous therapy, for example for several weeks or more, or for months or years. The above ranges are given as non-limiting examples, and it can be necessary in some embodiments to use doses outside of the recited ranges.

As an additional example, when olanzapine is administered in combination with zonisamide, preferred dosage forms are 5 mg olanzapine/60 mg zonisamide, and 10 mg olanzapine/120 mg zonisamide, generally with an olanzapine/zonisamide ratio of 1:12. For an admixture of risperidone and zonisamide, preferred dosage forms are 0.5 mg risperidone/30 mg zonisamide, 1 mg risperidone/60 mg zonisamide, and 2 mg risperidone/120 mg zonisamide with a risperidone/zonisamide ratio of 1:60. As yet another example, the daily dosage range for ziprasidone, for an oral dose, is in the range of about 20 mg to about 100 mg per day. In some embodiments, when ziprasidone is administered in combination with zonisamide, preferred dosage forms are 20 mg ziprasidone/60 mg zonisamide, and 40 mg ziprasisdone/120 mg zonisamide. However, the above ranges are given as non-limiting examples, and it can be necessary in some embodiments to use doses outside of the recited ranges.

Dosage amounts and intervals for the compositions disclosed herein can be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions comprising a compound disclosed herein formulated in a compatible pharmaceutical carrier can also be prepared, placed in an appropriate container, and labeled for treatment of an indicated condition.

In another aspect, provided herein are methods of treating a psychotic disorder comprising identifying a patient suffering from a psychotic disorder, and administering to the patient a first ingredient and a second ingredient, wherein the first ingredient and the second ingredient are as described above. As described above, the combination of the first and second ingredients has an enhanced efficacy in the treatment of psychotic disorders and/or their associated symptoms. In some embodiments, the first ingredient exerts a synergistic effect with the second ingredient with regard to the treatment of a psychotic disorder and/or symptoms related to the psychotic disorder.

In another aspect, provided herein are methods of enhancing the efficacy of an existing course of treatment with one or more antipsychotic agents, comprising identifying a patient subject to ongoing treatment with at least one antipsychotic agent, and administering to the patient, in addition to the existing course of treatment, the second ingredient, as described above.

In another aspect, provided herein are methods of treating a psychotic disorder in an overweight or obese patient comprising identifying a patient with a BMI greater than 25, and administering to the patient a first ingredient and a second ingredient, wherein the first ingredient and the second ingredient are as described above. In other embodiments, the individual has a BMI greater than 30. In still other embodiments, the individual has a BMI greater than 40. In other aspects, the methods involve treatment of individuals suffering from psychotic disorders regardless of body mass index.

In another aspect, provided herein are methods for treating one or more symptoms associated with a psychotic disorder, comprising identifying a patient suffering from a psychotic disorder associated with one or more symptoms in need of treatment, and administering to the patient a first ingredient and a second ingredient, wherein the first ingredient and the second ingredient are as described above.

In another aspect, provided herein are methods of stabilizing the mood of a patient suffering from a psychotic disorder, comprising identifying a patient suffering from a psychotic disorder in need of mood stabilization, and administering to the patient a first ingredient and a second ingredient, wherein the first and second ingredients are as described above.

In various embodiments, the psychotic disorder of the above methods is selected from the group consisting of bipolar disorders, schizophrenia, borderline personality, schizoid/schizotypal/paranoid personality disorders, delusional disorder, brief reactive psychosis, schizoaffective disorder, schizophreniform disorder, psychotic major depression, psychosis due to substance abuse, psychosis associated with disorders of development, and psychoses associated with medical conditions e.g., dementia, delirium, mental retardation etc.

In a further aspect, provided herein are methods of improving overall health outcomes, decreasing morbidity rates (e.g., through a reduction in suicidality, an outcome often associated with psychosis, mood disorders, or an interaction of both), or decreasing mortality rates in patients suffering from psychotic disorders, symptoms associated with psychotic disorders, and/or side effects associated with the treatment of a psychotic disorder. Overall health outcomes are determined by various means in the art. For example, improvements in morbidity and/or mortality rates, improvements in the patient's general feelings, improvements in the quality of life, improvements in the level of comfort at the end of life, and the like, are considered when overall health outcome are determined. Mortality rate is the number of patients who die while undergoing a particular treatment for a period of time compared to the overall number of patients undergoing the same or similar treatment over the same period of time. Morbidity rates are determined using various criteria, such as the frequency of hospital stays, the length of hospital stays, the frequency of visits to the doctor's office, the dosage of the medication being administered, and the like.

In various embodiments, the first ingredient and second ingredient are administered more or less simultaneously. In other embodiments the first ingredient is administered prior to the second ingredient. In yet other embodiments, the first ingredient is administered subsequent to the second ingredient. In certain embodiments, the first ingredient and the second ingredient are administered individually. In some embodiments, the first ingredient and the second ingredient are in separate administrable compositions, but the patient is directed to take the separate compositions nearly simultaneously, i.e., one pill is taken right after the other or one injection of one compound is made right after the injection of another compound, etc. In other embodiments the administering step comprises administering either the first ingredient or the second ingredient first and then administering the other one of either the first ingredient or the second ingredient. In these embodiments, the patient can be administered a composition comprising one of the ingredients and then at some time, e.g., a few minutes or a few hours later, be administered another composition comprising the other one of the ingredients. Also included in these embodiments are those in which the patient is administered a composition comprising one of the ingredients on a routine or continuous basis while receiving a composition comprising the other ingredient occasionally. In further embodiments, the patient can receive both ingredients on a routine or continuous basis, such a continuous infusion of the compound through an IV line.

In other embodiments, the first ingredient and the ingredient are in the same administrable composition, i.e., a single tablet, pill, or capsule, or a single solution for intravenous injection, or a single drinkable solution, or a single dragee formulation or patch, containing both compounds. In some embodiments, the first ingredient and the second ingredient are covalently linked to each other such that they form a single chemical entity. The single chemical entity is then digested and is metabolized, such as by enzymatic action, acid hydrolysis, base hydrolysis, or the like, into two separate physiologically active chemical entities one of which is the first ingredient and the other of which is the second ingredient. Advantageously, the combination of the first ingredient and second ingredient in the same administrable composition enhances the efficacy of the compositions and methods disclosed herein by improving patient compliance.

In certain embodiments, the patient can be a mammal. The mammal can be selected from the group consisting of mice, rats, rabbits, guinea pigs, dogs, cats, sheep, goats, cows, primates, such as monkeys, chimpanzees, and apes, and humans. In some embodiments, the patient is a human.

The compositions and methods disclosed herein are applicable to any psychotic disorder amenable to treatment, including but not limited to, schizophrenia, schizoaffective disorder, schizophreniform disorder, borderline personality disorder, delusional disorder, brief reactive psychosis, bipolar disorder, clinical depression, psychotic major depression, psychosis due to substance abuse, and psychoses associated with medical conditions e.g., senile dementia, Alzheimer's dementia, delirium, etc.

Some Embodiments of the Invention

Some of the embodiments of the present invention are as follows:

In the first embodiment, the invention relates to a composition for treating a psychotic disorder comprising a first ingredient and a second ingredient, wherein the first ingredient comprises at least one antipsychotic agent and the second ingredient comprises at least one anticonvulsant. Preferably, the composition does not include a combination of olanzapine, zonisamide, valproate and bupropion. Preferably, the composition does not include a combination of risperidone, zonisamide and paroxetine.

In the second embodiment, the invention relates to the composition of the first embodiment, wherein the at least one antipsychotic agent is selected from the group consisting of: chlorpromazine, fluphenazine, haloperidol, molindone, thiothixene, thioridazine, trifluoperazine, and loxapine.

In the third embodiment, the invention relates to the composition of the first embodiment, wherein the at least one antipsychotic agent is selected from the group consisting of: olanzapine (e.g., Zyprexa®), risperidone (e.g., Risperdal®), quetiapine (e.g., Seroquel®), ziprasidone (e.g., Geodon®), aripiprazole (e.g., Abilify®), and sertindole (e.g., Serdolect®).

In the fourth embodiment, the invention relates to the composition of the first embodiment, wherein the at least one antipsychotic agent is risperidone.

In the fifth embodiment, the invention relates to the composition of the first embodiment, wherein the at least one antipsychotic agent is olanzapine.

In the sixth embodiment, the invention relates to the composition of the first embodiment, wherein the at least one antipsychotic agent is selected from the group consisting of: lithium, valproate, carbamezepine, oxycarbamezepine, lamotrogine, tiagabine, and benzodiazepines.

In the seventh embodiment, the invention relates to the composition of the first embodiment, wherein the at least one anticonvulsant comprises a compound of structural Formula (I) as described above.

In the eighth embodiment, the invention relates to the composition of the seventh embodiment, wherein the compound of structural Formula (I) is zonisamide.

In the ninth embodiment, the invention relates to the composition of the first embodiment, wherein the at least one anticonvulsant comprises a compound of structural Formula (II) as described above.

In the tenth embodiment, the invention relates to the composition of the ninth embodiment, wherein the compound of structural Formula (II) is topiramate.

In the eleventh embodiment, the invention relates to the composition of the first embodiment, wherein the at least one anticonvulsant is selected from the group consisting of: zonisamide, topiramate, nembutal, lorazepam, clonazepam, clorazepate, tiagabine, gabapentin, fosphenyloin, phenyloin, carbamazepine, valproate, felbamate, levetiracetam, oxcarbazepine, lamotrigine, methsuximide, and ethosuxmide.

In the twelfth embodiment, the invention relates to the composition of the first embodiment, wherein the at least one anticonvulsant is a weight-loss promoting anticonvulsant selected from the group consisting of: compounds of structural Formula (I), zonisamide, compounds of structural Formula (II), topiramate, nembutal, lorazepam, clonazepam, clorazepate, tiagabine, gabapentin, fosphenyloin, phenyloin, carbamazepine, valproate, felbamate, levetiracetam, oxcarbazepine, lamotrigine, methsuximide, and ethosuxmide

In the thirteenth embodiment, the invention relates to the composition of the first embodiment, wherein the second ingredient further comprises an antidepressant.

In the fourteenth embodiment, the invention relates to the composition of the thirteenth embodiment, wherein the antidepressant is a selective serotonin reuptake inhibitor.

In the fifteenth embodiment, the invention relates to the composition of the fourteenth embodiment, wherein the selective serotonin reuptake inhibitor is selected from the group consisting of: fluoxetine, fluvoxamine, sertraline, paroxetine, citalopram, and escitalopram.

In the sixteenth embodiment, the invention relates to the composition of the thirteenth embodiment, wherein the antidepressant is a tricyclic antidepressant.

In the seventeenth embodiment, the invention relates to the composition of the sixteenth embodiment, wherein the tricyclic antidepressant is selected from the group consisting of: imipramine, desipramine, trimipramine, nortriptyline, clomipramine, doxepin, amitriptyline, maprotiline, protriptyline, dothiapen, and maprotiline.

In the eighteenth embodiment, the invention relates to the composition of the thirteenth embodiment, wherein the antidepressant is a MAO inhibitor.

In the nineteenth embodiment, the invention relates to the composition of the eighteenth embodiment, wherein the MAO inhibitor is selected from the group consisting of: phenelzine (e.g., Nardil®), tranylcypromine (e.g., Parnate®), isocarboxazid (e.g., Marplan®) and moclobemide (e.g., Aurorix®).

In the twentieth embodiment, the invention relates to the composition of the thirteenth embodiment, wherein the antidepressant is selected from the group consisting of: duloxetine, venlafaxine, nefazodone, mianserin setiptiline, viqualine trazodone, cianopramine, and mirtazapine.

In the twenty-first embodiment, the invention relates to the composition of the thirteenth embodiment, wherein the antidepressant is a compound that enhances the activity of norepinephrine and/or dopamine.

In the twenty-second embodiment, the invention relates to the composition of the twenty-first embodiment, wherein the compound that enhances the activity of norepinephrine and/or dopamine is selected from the group consisting of: atomoxetine, bupropion, thionisoxetine, and reboxetine.

In the twenty-third embodiment, the invention relates to the composition of the twenty-second embodiment, wherein the compound that enhances the activity of norepinephrine and/or dopamine is bupropion.

In the twenty-fourth embodiment, the invention relates to the composition of the first embodiment, wherein the first ingredient is risperidone and the second ingredient is zonisamide.

In the twenty-fifth embodiment, the invention relates to the composition of the first embodiment, wherein the first ingredient is risperidone and the second ingredient is topiramate.

In the twenty-sixth embodiment, the invention relates to the composition of the first embodiment, wherein the first ingredient is olanzapine and the second ingredient is zonisamide.

In the twenty-seventh embodiment, the invention relates to the composition of the first embodiment, wherein the first ingredient is olanzapine and the second ingredient is topiramate.

In the twenty-eighth embodiment, the invention relates to the composition of the thirteenth embodiment, wherein the first ingredient is risperidone, the second ingredient is zonisamide, and the antidepressant is bupropion.

In the twenty-ninth embodiment, the invention relates to the composition of the thirteenth embodiment, wherein the first ingredient is risperidone, the second ingredient is topiramate, and the antidepressant is bupropion.

In the thirtieth embodiment, the invention relates to the composition of the thirteenth embodiment, wherein the first ingredient is olanzapine, the second ingredient is zonisamide, and the antidepressant is bupropion.

In the thirty-first embodiment, the invention relates to the composition of the thirteenth embodiment, wherein the first ingredient is olanzapine, the second ingredient is topiramate, and the antidepressant is bupropion.

In the thirty-second embodiment, the invention relates to a method of treating a psychotic disorder comprising administering to a patient in need of treatment a first ingredient and a second ingredient, wherein the first ingredient comprises at least one antipsychotic agent and the second ingredient comprises at least one anticonvulsant. Preferably, olanzapine, zonisamide, valproate and bupropion are not simultaneously administered to the patient. Preferably, risperidone, zonisamide and paroxetine are not simultaneously administered to the patient.

In the thirty-third embodiment, the invention relates to a method of minimizing one or more side effects associated with the administration of a antipsychotic agent for the treatment of a psychotic disorder, comprising administering to a patient in need of treatment a first ingredient and a second ingredient, wherein the first ingredient comprises at least one antipsychotic agent and the second ingredient comprises at least one anticonvulsant. Preferably, olanzapine, zonisamide, valproate and bupropion are not simultaneously administered to the patient. Preferably, risperidone, zonisamide and paroxetine are not simultaneously administered to the patient.

In the thirty-fourth embodiment, the invention relates to a method of stabilizing the mood of a patient suffering from a psychotic disorder comprising identifying a patient suffering from a psychotic disorder that is in need of mood stabilization, and administering to the patient a first ingredient and second ingredient, wherein the first ingredient comprises at least one antipsychotic agent and the second ingredient comprises at least one anticonvulsant. Preferably, olanzapine, zonisamide, valproate and bupropion are not simultaneously administered to the patient. Preferably, risperidone, zonisamide and paroxetine are not simultaneously administered to the patient.

In the thirty-fifth embodiment, the invention relates to a method of enhancing the efficacy of an existing course of treatment with an antipsychotic agent comprising identifying a patient receiving ongoing treatment with an antipsychotic agent, and administering to the patient, in addition to the antipsychotic agent being administered on an ongoing basis, which comprises the first ingredient, a second ingredient comprising at least one anticonvulsant. wherein the first ingredient comprises at least one antipsychotic agent and the second ingredient comprises at least one anticonvulsant. Preferably, olanzapine, zonisamide, valproate and bupropion are not simultaneously administered to the patient. Preferably, risperidone, zonisamide and paroxetine are not simultaneously administered to the patient.

In the thirty-sixth embodiment, the invention relates to a method of treating one or more symptoms associated with a psychotic disorder, comprising administering to a patient in need of treatment a first ingredient and a second ingredient, wherein the first ingredient comprises at least one antipsychotic agent and the second ingredient comprises at least one anticonvulsant. wherein the first ingredient comprises at least one antipsychotic agent and the second ingredient comprises at least one anticonvulsant. Preferably, olanzapine, zonisamide, valproate and bupropion are not simultaneously administered to the patient. Preferably, risperidone, zonisamide and paroxetine are not simultaneously administered to the patient.

In the thirty-seventh embodiment, the invention relates to the method of the thirty-sixth embodiment, wherein the one or more symptoms associated with a psychotic disorder are selected from the group consisting of: hallucinations, delusions, mania, hypomania, aggression, paranoia, impairments in auditory or visual perception, confusion, ataxis, mood disorders, suicidality, and depression.

In the thirty-eighth embodiment, the invention relates to any of the methods of any of the thirty-second to the thirty-seventh embodiments, wherein the psychotic disorder is selected from the group consisting of: schizophrenia, schizoaffective disorder, schizophreniform disorder, borderline personality disorder, delusional disorder, brief reactive psychosis, bipolar disorder, clinical depression, psychotic major depression, psychosis due to substance abuse, and psychoses associated with medical conditions (e.g., senile dementia, Alzheimer's dementia, and delirium).

In the thirty-ninth embodiment, the invention relates to the methods of any of the thirty-second to the thirty-eighth embodiments, wherein the patient has a BMI greater than 25.

In the fortieth embodiment, the invention relates to the methods of any of the thirty-second through the thirty-eighth embodiments, wherein the patient has a BMI greater than 30.

In the forty-first embodiment, the invention relates to the methods of any of the thirty-second through the fortieth embodiments, wherein the first ingredient and second ingredient are administered substantially simultaneously.

In the forty-second embodiment, the invention relates to the methods of any of the thirty-second through the fortieth embodiments, wherein the first ingredient is administered prior to the second ingredient.

In the forty-third embodiment, the invention relates to the methods of any of the thirty-second through the fortieth embodiments, wherein the second ingredient is administered prior to the first ingredient.

In the forty-fourth embodiment, the invention relates to the methods of any of the thirty-second through the forty-first embodiments, wherein the first ingredient and second ingredient are administered as any of the compositions of the first through the thirty-first embodiments.

In the forty-fifth embodiment, the invention relates to the methods of any of the thirty-second through the forty-third embodiments, wherein the first ingredient is as defined in the compositions of any of the second through sixth embodiments.

In the forty-sixth embodiment, the invention relates to the methods of any of the thirty-second through the forty-third embodiments, wherein the second ingredient is as defined in the compositions of any of the seventh through twenty-third embodiments.

In the forty-seventh embodiment, the invention relates to the methods of any of the thirty-second through the forty-third embodiments, wherein the first and second ingredients are as defined in the compositions of any of the twenty-fourth through thirty-first embodiments.

In the forty-eighth embodiment, the invention relates to the methods of any of the thirty-second through the forty-seventh embodiments, wherein the plasma concentration levels of the first and second ingredients follow a similar time profile.

### EXAMPLES

The examples below are non-limiting and are merely representative of various aspects of the invention.

The prefrontal cortex in the brain is implicated in psychological disorders including schizophrenia and bipolar disorder. Similarly, the hypothalamus is implicated in mood disorders. Monoamine compounds include dopamine, serotonin and norepinephrine, and dopamine are thought to have a crucial role in arousal, emotion and cognition. Drugs that modify the synthesis and rate of release of monoamines, as well as their effects on the target tissues, are used to treat psychiatric disorders such as anxiety, depression and schizophrenia. By way of example, atypical antipsychotics, such as olanzapine, increase the release of dopamine and norepinepherine and have positive effects in treating psychological disorders. Serotonin antagonism is another property of atypical antipsychotics. Other drugs such as serotonin reuptake inhibitors and monoamine oxidase inhibitors, which result in effective increases in the concentration of monoamines in the brain are correlated with positive effects on psychological disorders (e.g., mood enhancement, improvement in cognitive performance, reduction in impulsivity).

Examples 1-4 below describe experiments to determine the in vivo concentration of monoamines (serotonin (5HT-2), dompamine (DA), and norepinephrine (NE)) in both the medial prefrontal cortex and the hypothalamus following treatment with various combinations of antipsychotics and anticonvulsants as a measure of the efficacy of the treatment regimen. Examples 5-8 describe protocols for using various combinations of antipsychotics and anticonvulsants. Example 9 describes treatment of obese individuals with any of the protocols exemplified in Examples 5-8.

Example 1, below, describes procedures to implant brain guide cannulae and/or microdialysis probes in rodents in order to perform microdialysis experiments.

### Example 1

### Implantation of Guide Cannulae and/or Brain Micordialysis Probes into Adult Male Rats

62 adult Sprague-Dawley male rats (Harland, Indianapolis) weighing 300-350 g were used in the following studies. The rats were quarantined for at least five days in group housing. Following the quarantine procedure, rats were maintained in individual cages. For surgical implantation with intracerebral guides, guides were inserted directly above either the hypothalamus (HT) (31 rats), or the medial prefrontal cortex (mPFC) (31 rats). Stereotaxic coordinates (Paxinos and Watson, 1986) provided below were used to position the guide cannulae and/or probes:

Stereotaxic coordinates

| mPFC | HT |
|---|---|
| anterior/posterior = +3.2 mm | anterior/posterior = 1.5 mm |
| lateral/medial = 0.8 mm | lateral/medial = 1.3 mm |
| dorsal/ventral = −1.4 mm | dorsal/ventral = −7.2 mm |
| extends to 4.7 mm from dura | extends to 9.0 mm from dura |

Animals were anesthetiszed according using standard procedures. The head of each animal was shaved from the front of the eyes to the back of the skull. Shaved areas were disinfected, and the animals were placed in stereotaxic frame ear bars. The animal was aligned with the incisor bar. The animal's scalp was cut with a sharp #15 scalpel blade. If the bone began to bleed, bone wax was applied to the incision. Periosteal tissue was cleaned from the skull to the lateral ridges with a cotton swab, and clamps were used to pull the skin out of the way. The probe or guide was placed in the clamp, such that the angled outlet cannula of the microdialysis probe was angled towards the animal's tail. The target coordinates were calculated, and this point was marked on the skull.

Two holes in opposing skull bones were drilled for bone anchor screws and thread screws. The dura was torn away with a sharp, pointed object. The guide cannulae were placed at the dorsal/ventral zero point. The guide cannulae were stereotaxically lowered into the brain from the dorsal/ventral point to the relative dorsal/ventral probe target. Using dental acrylic, the cannulae were cemented to the bone anchor screws. The cement was allowed to harden and the rodent was removed from the stereotaxic frame. The cranial and caudal aspects of the incision were sutured.

The animals were allowed to recover for three to five days. Stylets in the guides were replaced with dialysis probes on the evening prior to study to allow for animal acclimation and to reestablish the integrity of the blood brain barrier. To be accepted for the studies in the following examples, rats had to fall within 7% of pre-surgical weight, show no signs of clinical disease, and exhibit normal water and food consumption. Rats were weighed pre-surgery, every 1-2 days post-surgery until time of microdialysis, and postmortem.

The following example details the microdialysis protocol used in the studies described in Examples 3 and 4.

### Example 2

### Microdialysis Study

Microdialysis probes used in the experiments described below were first soaked in standard Ringer's perfusion medium for 30 minutes. Inlet and outlet tubing was connected to each probe using flanged connectors. The outlet tubing was connected to a fraction collector, and the inlet was connected to and Empris syringe drive. The probes were immersed in fresh Ringer's solution and flushed with Ringer's perfusion medium at a rate of 2 μl/min for 1 hour. The probe was then transferred to the intracerebral guide on the rat's skull.

The following formulations were used to administration in the microdialysis experiments described below.

Olanzapine was administered to rats at a final concentration of 1 mg/kg intraperitoneally. 2.1 ml of water was added to a single vial of ZYPREXA® (containing 11.0 mg olanzapine in powder form), and the vial was rotated until the contents dissolved. Water was added to obtain a final concentration of 0.3 mg/ml.

Ziprasidone was administered to rats at a final concentration of 3 mg/kg intraperitoneally. 1.2 ml of water was added to a single vial of GEODON® (containing 20 mg ziprasidone and 4.7 mg methanesulfonic acid solubilized by 294 mg of sulfobutylether b-cyclodextrin sodium). Water was added to obtain a final concentration of 3 mg/ml ziprasidone.

Zonisamide was administered to rats in a final concentration of 25 mg/kg intraperitoneally in a vehicle of 13.4% EtOH, 20.1% propylene glycol, 66.5% saline. Zonisamide was dissolved in DMSO. The dissolved zonisamide was combined with vehicle solution that had been heated to 60°-90° C. at a final concentration of 10%. The final concentration of zonisamide was 7.5 mg/ml. The drug solution was maintained at 37° C. prior to injection.

The rats were divided into ten test groups. Five animals were analyzed for each test group. The test groups were as follows:

1: Single IP dose of zonisamide; collection of dialysates from hypothalamus (n=5)

2. Single IP dose of zonisamide; collection of dialysates from mPFC (n=5)

3. Single IP dose of olanzapine, 1 mg/kg; collection of dialysates from hypothalalmus (n=5)

4. Single IP dose of olanzapine, 1 mg/kg; collection of dialysates from mPFC (n=5)

5. Single IP dose of ziprasidone, 3 mg/kg; collection of dialysates from hypothalamus (n=5)

6. Single IP dose of ziprasidone, 3 mg/kg; collection of dialysates from mPFC (n=5)

7. Single IP dose of combination zonisamide and olanzapine, 1 mg/kg; collection of dialysates from hypothalamus (n=5)

8 Single IP dose of combination zonisamide and olanzapine, 1 mg/kg; collection of dialysates from mPFC (n=5)

9. Single IP dose of combination zonisamide and ziprasidone, 3 mg/kg; collection of dialysates from hypothalamus (n=5)

10. Single IP dose of combination zonisamide and ziprasidone, 3 mg/kg; collection of dialysates from mPFC (n=5)

For the microdialysis experiments, perfusion consisted of 2 μl/min delivery of sterile standard Ringer's solution for intervals of 20 minutes. Final collection volumes were 40 μl. 30 μl samples were analyzed for each analyte: DA, NE, 5HT. 12 pre-dose samples and 12 post dose samples were collected every 20 minutes for a duration of 4 hours, both pre-dose and post-dose. Six samples from the pre-dose and post-dose collections were analyzed for norepinephrine. The remaining six samples from the pre-dose and post-dose collections were analyzed for both dopamine and serotonin.

Samples were chilled to −80° C. immediately following collection. The samples were analyzed using liquid chromatography with electrochemical detection using conventional techniques. See, e.g., Huang, T., R. et al. (1994) New SepStik Microbore Columns for Liquid Chromatography. Current Separations 12(4): 191-195.

The following example demonstrates that the combination of zonisamide and ziprasidone synergistically affects the levels of dopamine, norepinephrine and serotonin in the brain.

### Example 3

### The Combination of Ziprasidone and Zonisamide Provide an Unexpected Increase in Monoamines within the Brain

Study groups 1, 2, 5, 6, 9 and 10, discussed in Example 2 were used to evaluate the efficacy of the combination of ziprasidone with zonisamide. Concentrations of each compound are expressed as % baseline. The baseline numbers were determined by averaging the concentration of the monoamine compound (i.e., 5-HT2, DA, NE) at the three timepoints prior to the addition of the test substance (t=0). The data from the experiments are presented in Tables 1-6, below. Each data point represents the average of the values from the 5 animals in the study group.

TABLE 1
SEROTONIN CONCENTRATIONS IN HYPOTHALAMUS

| | | | | | | Serotonin concentrations in | | |
|---|---|---|---|---|---|---|---|---|
| Serotonin concentrations in | | | Serotonin concentrations in | | | hypothalamus - 35 mg/kg | | |
| hypothalamus - | | | hypothalamus - | | | zonisamide + 3 mg/kg | | |
| 25 mg/kg zonisamide | | | 3 mg/kg ziprasidone | | | ziprasidone | | |
| Time | | | Time | | | Time | | |
| (minutes | | | (minutes | | | (minutes | | |
| pre/post | | Std. | pre/post | | Std. | pre/post | | Std. |
| dose) | Concentration | Error | dose) | Concentration | Error | dose) | Concentration | Error |
| −100 | 87.552 | 11.860 | −100 | 84.644 | 12.243 | −100 | 111.156 | 4.043 |
| −60 | 79.388 | 15.323 | −60 | 109.552 | 8.847 | −60 | 98.233 | 2.345 |
| −20 | 133.061 | 14.285 | −20 | 105.804 | 3.904 | −20 | 90.611 | 5.169 |
| +40 | 139.708 | 22.248 | +40 | 147.484 | 25.368 | +40 | 85.461 | 4.178 |
| +80 | 88.171 | 28.407 | +80 | 131.609 | 25.071 | +80 | 86.365 | 3.450 |
| +120 | 87.586 | 18.774 | +120 | 122.447 | 15.114 | +120 | 108.321 | 10.591 |
| +160 | 64.925 | 15.218 | +160 | 127.006 | 13.490 | +160 | 130.926 | 13.333 |
| +200 | 98.920 | 21.015 | +200 | 140.594 | 20.922 | +200 | 123.946 | 15.783 |
| +220 | 100.672 | 22.805 | +220 | 88.071 | 12.338 | +220 | 134.542 | 10.232 |
| 3 HOUR | | | 3 HOUR | | | 3 HOUR | | |
| AVE | 96.664 | 22.304 | AVE | 122.083 | 12.994 | AVE | 110.216 | 5.075 |

TABLE 2
SEROTONIN CONCENTRATIONS IN MPFC

| Serotonin concentrations | | | Serotonin concentrations | | | Serotonin concentrations in | | |
|---|---|---|---|---|---|---|---|---|
| in mPFC - | | | in mPFC - | | | mPFC - 35 mg/kg zonisamide + | | |
| 25 mg/kg zonisamide | | | 3 mg/kg ziprasidone | | | 3 mg/kg ziprasidone | | |
| Time | | | Time | | | Time | | |
| (minutes | | | (minutes | | | (minutes | | |
| pre/post | | Std. | pre/post | | Std. | pre/post | | Std. |
| dose) | Concentration | Error | dose) | Concentration | Error | dose) | Concentration | Error |
| −100 | 72.700 | 14.232 | −100 | 95.050 | 9.827 | −100 | 97.154 | 5.262 |
| −60 | 119.648 | 10.894 | −60 | 101.254 | 8.084 | −60 | 112.962 | 14.949 |
| −20 | 107.652 | 7.597 | −20 | 103.695 | 11.643 | −20 | 89.884 | 14.899 |
| 40 | 96.932 | 18.727 | 40 | 100.119 | 19.398 | 40 | 116.938 | 14.591 |
| 80 | 74.461 | 15.041 | 80 | 80.736 | 12.373 | 80 | 125.584 | 20.264 |
| 120 | 60.557 | 9.911 | 120 | 80.678 | 16.996 | 120 | 123.105 | 31.426 |
| 160 | 82.729 | 17.152 | 160 | 76.032 | 8.806 | 160 | 117.177 | 14.961 |
| 200 | 89.529 | 13.174 | 200 | 96.412 | 24.413 | 200 | 121.773 | 28.142 |
| 240 | 63.518 | 12.595 | 240 | 82.843 | 12.648 | 240 | 129.679 | 27.355 |
| 3 HOUR | 77.954 | 14.676 | 3 HOUR | 88.108 | 11.819 | 3 HOUR | 123.842 | 19.041 |
| AVE | | | AVE | | | AVE | | |

TABLE 3
DOPAMINE CONCENTRATIONS IN HYPOTHALAMUS

| Dopamine concentrations in | | | Dopamine concentrations in | | | Dopamine concentrations in | | |
|---|---|---|---|---|---|---|---|---|
| hypothalamus - 25 mg/kg | | | hypothalamus - 3 mg/kg | | | hypothalamus - 35 mg/kg zonisamide + | | |
| zonisamide | | | ziprasidone | | | 3 mg/kg ziprasidone | | |
| Time | | | Time | | | Time | | |
| (minutes | | | (minutes | | | (minutes | | |
| pre/post | | Std. | pre/post | | Std. | pre/post | | |
| dose) | Concentration | Error | dose) | Concentration | Error | dose) | Concentration | Std. Error |
| −100 | 101.354 | 35.498 | −100 | 93.334 | 3.129 | −100 | 104.493 | 8.642 |
| −60 | 56.252 | 16.568 | −60 | 110.026 | 10.274 | −60 | 113.266 | 8.149 |
| −20 | 142.394 | 26.936 | −20 | 96.640 | 12.835 | −20 | 82.240 | 5.723 |
| 40 | 121.635 | 36.193 | 40 | 155.264 | 44.988 | 40 | 460.202 | 50.155 |
| 80 | 72.946 | 18.859 | 80 | 133.547 | 25.074 | 80 | 326.536 | 41.303 |
| 120 | 91.707 | 25.446 | 120 | 119.303 | 18.705 | 120 | 235.420 | 39.505 |
| 160 | 66.181 | 12.755 | 160 | 108.156 | 17.413 | 160 | 201.917 | 32.433 |
| 200 | 143.010 | 54.661 | 200 | 114.436 | 13.854 | 200 | 173.935 | 24.721 |
| 240 | 123.012 | 57.497 | 240 | 96.372 | 15.614 | 240 | 148.705 | 16.349 |
| 3 HOUR | | | 3 HOUR | | | 3 HOUR | | |
| AVE | 105.438 | 37.4503 | AVE | 122.276 | 14.924 | AVE | 257.786 | 28.4163 |

TABLE 4
DOPAMINE CONCENTRATIONS IN MPFC

| Dopamine concentrations | | | Dopamine concentrations | | | Dopamine concentrations in | | |
|---|---|---|---|---|---|---|---|---|
| in mPFC - | | | in mPFC - | | | mPFC - 35 mg/kg zonisamide + | | |
| 25 mg/kg zonisamide | | | 3 mg/kg ziprasidone | | | 3 mg/kg ziprasidone | | |
| Time | | | Time | | | Time | | |
| (minutes | | | (minutes | | | (minutes | | |
| pre/post | | Std. | pre/post | | Std. | pre/post | | |
| dose) | Concentration | Error | dose | Concentration | Error | dose | Concentration | Std. Error |
| −100 | 113.677 | 23.340 | −100 | 106.240 | 8.293 | −100 | 99.259 | 13.997 |
| −60 | 87.652 | 14.788 | −60 | 88.131 | 4.294 | −60 | 93.711 | 4.794 |
| −20 | 98.672 | 16.631 | −20 | 105.629 | 6.769 | −20 | 107.029 | 9.923 |
| 40 | 166.688 | 22.367 | 40 | 224.637 | 29.019 | 40 | 477.365 | 170.492 |
| 80 | 86.224 | 11.815 | 80 | 220.406 | 31.948 | 80 | 286.610 | 57.597 |
| 120 | 111.764 | 16.760 | 120 | 204.006 | 43.645 | 120 | 226.615 | 63.790 |
| 160 | 109.627 | 12.516 | 160 | 155.426 | 25.034 | 160 | 206.180 | 35.452 |
| 200 | 94.060 | 21.854 | 200 | 144.584 | 26.359 | 200 | 187.734 | 25.334 |
| 240 | 134.732 | 27.073 | 240 | 109.557 | 22.289 | 240 | 156.819 | 31.160 |
| 3 HOUR | | | 3 HOUR | | | 3 HOUR | | |
| AVE | 117.183 | 21.5678 | AVE | 180.798 | 29.2319 | AVE | 263.641 | 58.5183 |

TABLE 5
NOREPINEPHRINE CONCENTRATION IN HYPOTHALAMUS

| | | | | | | Norepinephrine concentrations | | |
|---|---|---|---|---|---|---|---|---|
| Norepinephrine concentrations in | | | Norepinephrine concentrations in | | | in hypothalamus - 35 mg/kg | | |
| hypothalamus - 25 mg/kg | | | hypothalamus - 3 mg/kg | | | zonisamide + | | |
| zonisamide | | | ziprasidone | | | 3 mg/kg ziprasidone | | |
| Time | | | Time | | | Time | | |
| (minutes | | | (minutes | | | (minutes | | |
| pre/post | | Std. | pre/post | | Std. | pre/post | | |
| dose) | Concentration | Error | dose) | Concentration | Error | dose) | Concentration | Std. Error |
| −120 | 100.460 | 7.439 | −120 | 93.838 | 4.454 | −120 | 108.581 | 14.331 |
| −80 | 88.184 | 9.697 | −80 | 95.746 | 9.485 | −80 | 113.450 | 12.952 |
| −40 | 111.356 | 10.857 | −40 | 110.416 | 11.705 | −40 | 77.969 | 10.540 |
| 20 | 194.380 | 59.955 | 20 | 221.458 | 46.529 | 20 | 436.514 | 121.098 |
| 60 | 151.267 | 33.280 | 60 | 286.925 | 80.682 | 60 | 906.866 | 380.812 |
| 100 | 101.287 | 13.960 | 100 | 186.090 | 44.286 | 100 | 489.087 | 147.497 |
| 140 | 99.489 | 14.057 | 140 | 130.369 | 19.606 | 140 | 207.518 | 85.039 |
| 180 | 102.237 | 13.551 | 180 | 123.480 | 14.321 | 180 | 188.680 | 69.979 |
| 220 | 91.722 | 10.349 | 220 | 115.795 | 9.349 | 220 | 140.346 | 42.657 |
| 3 HOUR | | | 3 HOUR | | | 3 HOUR | | |
| AVE | 124.272 | 34.4224 | AVE | 182.01 | 35.3372 | AVE | 394.835 | 109.542 |

TABLE 6
NOREPINEPHRINE CONCENTRATION IN MPFC

| Norepinephrine concentrations | | | Norepinephrine concentrations | | | Norepinephrine concentrations | | |
|---|---|---|---|---|---|---|---|---|
| in mPFC - 25 mg/kg | | | in mPFC - 3 mg/kg | | | in mPFC - 35 mg/kg zonisamide + | | |
| zonisamide | | | ziprasidone | | | 3 mg/kg ziprasidone | | |
| Time | | | Time | | | Time | | |
| (minutes | | | (minutes | | | (minutes | | |
| pre/post | | Std. | pre/post | | Std. | pre/post | | |
| dose) | Concentration | Error | dose) | Concentration | Error | dose) | Concentration | Std. Error |
| −120 | 92.300 | 10.517 | −120 | 101.750 | 3.524 | −120 | 89.565 | 7.268 |
| −80 | 106.249 | 22.478 | −80 | 96.063 | 4.559 | −80 | 98.699 | 7.269 |
| −40 | 101.451 | 12.474 | −40 | 102.187 | 7.515 | −40 | 111.735 | 8.347 |
| 20 | 164.376 | 27.274 | 20 | 152.679 | 12.365 | 20 | 235.245 | 49.936 |
| 60 | 102.033 | 7.490 | 60 | 173.477 | 12.083 | 60 | 289.637 | 62.069 |
| 100 | 105.920 | 10.530 | 100 | 139.468 | 7.859 | 100 | 234.952 | 45.371 |
| 140 | 102.114 | 7.969 | 140 | 135.555 | 7.873 | 140 | 222.729 | 37.049 |
| 180 | 115.233 | 13.703 | 180 | 120.130 | 9.052 | 180 | 197.357 | 37.456 |
| 220 | 95.176 | 11.042 | 220 | 120.068 | 12.457 | 220 | 175.829 | 33.695 |
| 3 HOUR | | | 3 HOUR | | | 3 HOUR | | |
| AVE | 114.142 | 16.9984 | AVE | 140.23 | 8.84877 | AVE | 225.958 | 43.6912 |

The data above demonstrate that the combination of zonisamide and ziprasidone results in a synergistic increase in the concentration of serotonin and dopamine in the hypothalamus and medial prefrontal cortex compared to either compound alone. Further, the combination of zonisamide and ziprasidone caused a synergistic increase in the concentration of norepinephrine in the medial prefrontal cortex compared to either compound alone. The data is also presented in graphical form in FIGS. 1-6.

The following example demonstrates that the combination of zonisamide and olanzapine synergistically affects the levels of dopamine, norepinephrine and serotonin in the brain.

### Example 4

### The Combination of Olanzapine and Zonisamide Provides an Unexpected Increase in Monoamines within the Brain

Study groups 1, 2, 3, 4, 9 and 10, discussed in Example 2 were used to evaluate the efficacy of the combination of olanzapine with zonisamide. Concentrations of each compound are expressed as % baseline as described in Example 3. The data from the experiments are presented in Tables 7-12, below. Each data point represents the average of the values from the 5 animals in the study group.

TABLE 7
SEROTONIN CONCENTRATIONS IN HYPOTHALAMUS

| Serotonin concentrations in | | | Serotonin concentrations | | | Serotonin concentrations in | | |
|---|---|---|---|---|---|---|---|---|
| hypothalamus - 25 | | | in hypothalamus - | | | hyptothalamus - 35 mg/kg | | |
| mg/kg zonisamide | | | 3 mg/kg olanzapine | | | zonisamide + 3 mg/kg olanzapine | | |
| Time | | | Time | | | Time | | |
| (minutes | | | (minutes | | | (minutes | | |
| pre/post | | | pre/post | | | pre/post | | |
| dose) | Concentration | Std. Error | dose) | Concentration | Std. Error | dose) | Concentration | Std. Error |
| −100 | 87.552 | 11.860 | −100 | 93.959 | 12.477 | −100 | 91.979 | 7.707 |
| −60 | 79.388 | 15.323 | −60 | 98.601 | 10.697 | −60 | 91.298 | 9.437 |
| −20 | 133.061 | 14.285 | −20 | 107.440 | 21.958 | −20 | 116.723 | 11.865 |
| 40 | 139.708 | 22.248 | 40 | 89.816 | 17.401 | 40 | 144.709 | 31.841 |
| 80 | 88.171 | 28.407 | 80 | 86.183 | 11.644 | 80 | 102.662 | 30.454 |
| 120 | 87.586 | 18.774 | 120 | 92.131 | 25.198 | 120 | 104.657 | 28.016 |
| 160 | 64.925 | 15.218 | 160 | 80.560 | 8.582 | 160 | 115.130 | 18.740 |
| 200 | 98.920 | 21.015 | 200 | 98.707 | 10.260 | 200 | 107.784 | 13.201 |
| 240 | 100.672 | 22.805 | 240 | 88.921 | 6.044 | 240 | 117.182 | 21.944 |
| 3 HOUR | | | 3 HOUR | | | 3 HOUR | | |
| AVE | 96.6637 | 22.304 | AVE | 89.0647 | 13.5642 | AVE | 115.291 | 26.1856 |

TABLE 8
SEROTONIN CONCENTRATIONS IN MPFC

| Serotonin concentrations | | | Serotonin concentrations | | | Serotonin concentrations | | |
|---|---|---|---|---|---|---|---|---|
| in mPFC - | | | in mPFC - 3 mg/kg | | | in mPFC - 35 mg/kg | | |
| 25 mg/kg zonisamide | | | olanzapine | | | zonisamide + 3 mg/kg olanzapine | | |
| Time | | | Time | | | Time | | |
| (minutes | | | (minutes | | | (minutes | | |
| pre/post | | Std. | pre/post | | Std. | pre/post | | Std. |
| dose) | Concentration | Error | dose) | Concentration | Error | dose) | Concentration | Error |
| −100 | 72.700 | 14.232 | −100 | 100.565 | 12.084 | −100 | 109.387 | 12.110 |
| −60 | 119.648 | 10.894 | −60 | 100.982 | 12.939 | −60 | 104.229 | 13.337 |
| −20 | 107.652 | 7.597 | −20 | 98.453 | 10.055 | −20 | 86.384 | 11.689 |
| 40 | 96.932 | 18.727 | 40 | 115.609 | 22.278 | 40 | 137.635 | 30.498 |
| 80 | 74.461 | 15.041 | 80 | 101.724 | 19.920 | 80 | 127.468 | 22.949 |
| 120 | 60.557 | 9.911 | 120 | 88.189 | 15.889 | 120 | 113.149 | 15.468 |
| 160 | 82.729 | 17.152 | 160 | 92.085 | 19.433 | 160 | 77.058 | 8.003 |
| 200 | 89.529 | 13.174 | 200 | 119.171 | 19.784 | 200 | 88.170 | 4.771 |
| 240 | 63.518 | 12.595 | 240 | 83.828 | 13.183 | 240 | 69.922 | 9.967 |
| 3 HOUR | | 14.676 | 3 HOUR | | | 3 HOUR | | |
| AVE | 77.9543 | 7 | AVE | 88.1078 | 11.819 | AVE | 123.842 | 19.0411 |

TABLE 9
DOPAMINE CONCENTRATIONS IN HYPOTHALAMUS

| Dopamine concentrations in | | | Dopamine concentrations in | | | Dopamine concentrations in | | |
|---|---|---|---|---|---|---|---|---|
| hypothalamus - 25 mg/kg | | | hypothalamus - 3 mg/kg | | | hyptothalamus - 35 mg/kg | | |
| zonisamide | | | olanzapine | | | zonisamide + 3 mg/kg olanzapine | | |
| Time | | | Time | | | Time | | |
| (minutes | | | (minutes | | | (minutes | | |
| pre/post | | | pre/post | | | pre/post | | |
| dose) | Concentration | Std. Error | dose) | Concentration | Std. Error | dose) | Concentration | Std. Error |
| −100 | 101.354 | 35.498 | −100 | 106.697 | 21.574 | −100 | 80.316 | 7.418 |
| −60 | 56.252 | 16.568 | −60 | 93.473 | 12.460 | −60 | 123.778 | 13.112 |
| −20 | 142.394 | 26.936 | −20 | 99.830 | 29.458 | −20 | 95.906 | 9.441 |
| 40 | 121.635 | 36.193 | 40 | 156.507 | 39.736 | 40 | 258.711 | 51.057 |
| 80 | 72.946 | 18.859 | 80 | 110.363 | 23.618 | 80 | 172.365 | 28.922 |
| 120 | 91.707 | 25.446 | 120 | 183.338 | 48.433 | 120 | 141.239 | 19.158 |
| 160 | 66.181 | 12.755 | 160 | 89.032 | 23.538 | 160 | 241.239 | 80.965 |
| 200 | 143.010 | 54.661 | 200 | 113.293 | 41.948 | 200 | 128.435 | 18.595 |
| 240 | 123.012 | 57.497 | 240 | 95.959 | 18.112 | 240 | 143.869 | 8.721 |
| 3 HOUR | | | 3 HOUR | | | 3 HOUR | | |
| AVE | 104.3854 | 37.4503 | AVE | 125.98 | 34.9645 | AVE | 184.7645 | 45.7635 |

TABLE 10
Dopamine Concentrations in mPFC

| | | | | | | Dopamine concentrations | | |
|---|---|---|---|---|---|---|---|---|
| Dopamine concentrations | | | Dopamine concentrations | | | in mPFC - 35 mg/kg | | |
| in mPFC - | | | in mPFC - 3 mg/kg | | | zonisamide + 3 mg/kg | | |
| 25 mg/kg zonisamide | | | olanzapine | | | olanzapine | | |
| Time | | | Time | | | Time | | |
| (minutes | | | (minutes | | | (minutes | | |
| pre/post | | Std. | pre/post | | Std. | pre/post | | Std. |
| dose) | Concentration | Error | dose) | Concentration | Error | dose) | Concentration | Error |
| −100 | 113.677 | 23.340 | −100 | 75.482 | 5.223 | −100 | 102.079 | 7.112 |
| −60 | 87.652 | 14.788 | −60 | 119.009 | 7.735 | −60 | 100.906 | 7.042 |
| −20 | 98.672 | 16.631 | −20 | 105.509 | 9.271 | −20 | 97.015 | 4.869 |
| 40 | 166.688 | 22.367 | 40 | 227.023 | 42.090 | 40 | 264.374 | 39.517 |
| 80 | 86.224 | 11.815 | 80 | 266.655 | 49.768 | 80 | 208.503 | 35.654 |
| 120 | 111.764 | 16.760 | 120 | 219.135 | 25.867 | 120 | 200.101 | 24.600 |
| 160 | 109.627 | 12.516 | 160 | 186.256 | 26.079 | 160 | 196.659 | 35.878 |
| 200 | 94.060 | 21.854 | 200 | 202.788 | 25.211 | 200 | 174.865 | 17.199 |
| 240 | 134.732 | 27.073 | 240 | 153.048 | 22.386 | 240 | 155.344 | 23.097 |
| 3 HOUR | | | 3 HOUR | | | 3 HOUR | | |
| AVE | 117.183 | 21.5678 | AVE | 209.94 | 34.4321 | AVE | 199.974 | 31.6615 |

TABLE 11
NOREPINEPHRINE CONCENTRATIONS IN HYPOTHALAMUS

| Norepinephrine concentrations in | | | | | | Norepinephrine concentrations in | | |
|---|---|---|---|---|---|---|---|---|
| hypothalamus - 25 mg/kg | | | Norepinephrine concentrations in | | | hyptohalamus - 35 mg/kg zonisamide + | | |
| zonisamide | | | hypothalamus - 3 mg/kg olanzapine | | | 3 mg/kg olanzapine | | |
| Time | | | Time | | | Time | | |
| (minutes | | | (minutes | | | (minutes | | |
| pre/post dose) | Concentration | Std. Error | pre/post dose) | Concentration | Std. Error | pre/post dose) | Concentration | Std. Error |
| −120 | 100.460 | 7.439 | −120 | 91.097 | 8.529 | −120 | 93.036 | 3.916 |
| −80 | 88.184 | 9.697 | −80 | 103.272 | 7.796 | −80 | 95.893 | 8.858 |
| −40 | 111.356 | 10.857 | −40 | 105.631 | 12.064 | −40 | 111.071 | 6.813 |
| 20 | 194.380 | 59.955 | 20 | 161.934 | 28.858 | 20 | 451.300 | 146.624 |
| 60 | 151.267 | 33.280 | 60 | 148.422 | 28.127 | 60 | 398.470 | 130.511 |
| 100 | 101.287 | 13.960 | 100 | 115.418 | 20.655 | 100 | 224.243 | 61.883 |
| 140 | 99.489 | 14.057 | 140 | 118.651 | 25.117 | 140 | 204.540 | 57.513 |
| 180 | 102.237 | 13.551 | 180 | 113.533 | 15.628 | 180 | 164.165 | 46.622 |
| 220 | 91.722 | 10.349 | 220 | 115.014 | 16.729 | 220 | 239.447 | 73.910 |
| 3 HOUR AVE | 124.221 | 32.4224 | 3 HOUR AVE | 129.18 | 22.3442 | 3 HOUR AVE | 280.361 | 98.3424 |

TABLE 12
NOREPINEPHRINE CONCENTRATIONS IN MPFC

| | | | | | | Norepinephrine concentrations in | | |
|---|---|---|---|---|---|---|---|---|
| Norepinephrine concentrations in | | | Norepinephrine concentrations in | | | mPFC - 35 mg/kg zonisamide + | | |
| mPFC - 25 mg/kg zonisamide | | | mPFC - 3 mg/kg olanzapine | | | 3 mg/kg olanzapine | | |
| Time | | | Time | | | Time | | |
| (minutes | | | (minutes | | | (minutes | | |
| pre/post dose) | Concentration | Std. Error | pre/post dose) | Concentration | Std. Error | pre/post dose) | Concentration | Std. Error |
| −120 | 92.300 | 10.517 | −120 | 82.120 | 10.718 | −120 | 96.813 | 6.070 |
| −80 | 106.249 | 22.478 | −80 | 119.880 | 17.039 | −80 | 89.478 | 6.392 |
| −40 | 101.451 | 12.474 | −40 | 98.000 | 13.700 | −40 | 113.709 | 5.070 |
| 20 | 164.376 | 27.274 | 20 | 162.923 | 26.959 | 20 | 212.402 | 14.750 |
| 60 | 102.033 | 7.490 | 60 | 197.693 | 30.519 | 60 | 185.278 | 13.376 |
| 100 | 105.920 | 10.530 | 100 | 159.929 | 35.537 | 100 | 160.023 | 12.499 |
| 140 | 102.114 | 7.969 | 140 | 141.759 | 21.689 | 140 | 156.876 | 8.899 |
| 180 | 115.233 | 13.703 | 180 | 147.978 | 8.323 | 180 | 155.721 | 8.770 |
| 220 | 95.176 | 11.042 | 220 | 117.569 | 11.148 | 220 | 152.947 | 10.428 |
| 3 HOUR AVE | 114.112 | 16.998 | 3 HOUR AVE | 154.642 | 24.8268 | 3 HOUR AVE | 170.541 | 14.463 |

The data above demonstrate that the combination of zonisamide and olanzapine results in a synergistic increase in the concentration of serotonin in the hypothalamus and medial prefrontal cortex compared to either compound alone. Further, the combination of zonisamide and ziprasidone caused a synergistic increase in the concentration of dopamine and norepinephrine in the hypothalamus compared to either compound alone. The data is also presented in graphical form in FIGS. 1-6.

The following examples describe use of various combinations of antipsychotics and anticonvulsants for the treatment of individuals.

### Example 5

### Use of Zonisamide with Risperidone or Olanzapine

Individuals taking risperidone or olanzapine, or who are about to take risperidone or olanzapine, who have experienced side effects, such as weight gain, depression, or other mood disorders, as the result of the use of the antipsychotic agent, or who are susceptible to such side effects as the result of the use of the antipsychotic agent, are identified. Each individual is instructed to take one 25 mg tablet of zonisamide on a daily basis, in addition to the antipsychotic agent therapy.

The individuals are monitored for a period of months, with measurement of symptoms indicative of the efficacy of treatment of the underlying psychotic disorder and relevant side effects. The dosage is adjusted to minimize symptoms of the psychotic disorder and adverse side effects. In the case of weight gain, dosages are typically adjusted so that the patient loses weight at a rate of 10% of initial weight every 6 months. However, the rate of weight loss for each individual can be adjusted by the treating physician based on the individual's particular needs.

The dosage of zonisamide can be from about 25 mg to about 800 mg per day, generally given once per day or divided (e.g., equally) into multiple doses. Preferably, the dose is from about 100 mg to about 600 mg per day, more preferably, the dose is from about 200 mg to about 400 mg per day. Zonisamide tablets are usually made and marketed in 25 mg, 50 mg, and 100 mg doses. Risperidone is given in daily dosages of between about 0.1 mg and 10 mg, preferably between 1 mg and 5 mg, generally given once per day or divided (e.g., equally) into multiple doses. Risperidone is generally available in 0.25 mg, 0.5 mg, 1 mg, 2 mg, 3 mg, and 4 mg oral dosage units. Olanzapine is given in daily dosages of between about 5 mg and 30 mg, preferably between 5 mg and 15 mg, generally given once per day or divided (e.g., equally) into multiple doses. Olanzapine is typically available in doses of 2.5 mg, 5 mg, 10 mg, 15 mg, or 20 mg. Individual tablets, or combination of tablets can be used to achieve the desired dosing. In some instances, it may be necessary to use dosages outside these ranges.

### Example 6

### Use of Topiramate with Risperidone or Olanzapine

Individuals taking risperidone or olanzapine, or who are about to take risperidone or olanzapine, who have experienced side effects, such as weight gain, depression, or other mood disorders, as the result of the use of the antipsychotic agent, or who are susceptible to such side effects as the result of the use of the antipsychotic agent, are identified. Each individual is instructed to take one 25 mg tablet of topiramate on a daily basis, in addition to the antipsychotic agent therapy.

The individuals are monitored for a period of months, with measurement of symptoms indicative of the efficacy of treatment of the underlying psychotic disorder and relevant side effects. The dosage is adjusted to minimize symptoms of the psychotic disorder and adverse side effects. In the case of weight gain, dosages are typically adjusted so that the patient loses weight at a rate of 10% of initial weight every 6 months. However, the rate of weight loss for each individual can be adjusted by the treating physician based on the individual's particular needs.

The dosage of topiramate can be from about 25 mg to about 1600 mg, preferably from about 50 mg to about 600 mg, more preferably from about 100 mg to about 400 mg. Risperidone is given in daily dosages of between about 0.1 mg and 10 mg, preferably between 1 mg and 5 mg, generally given once per day or divided (e.g., equally) into multiple doses. Risperidone is generally available in 0.25 mg, 0.5 mg, 1 mg, 2 mg, 3 mg, and 4 mg oral dosage units. Olanzapine is most often given in daily dosages of between about 5 mg and 30 mg, preferably between 5 mg and 15 mg, generally given once per day or divided (e.g., equally) into multiple doses. Olanzapine is typically available in doses of 2.5 mg, 5 mg, 10 mg, 15 mg, or 20 mg. Individual tablets, or combination of tablets can be used to achieve the desired dosing. In some instances, it may be necessary to use dosages outside these ranges.

### Example 7

### Combination of Zonisamide or Topiramate and Bupropion with Risperidone or Olanzapine

Individuals taking risperidone or olanzapine, or who are about to take risperidone or olanzapine, who have experienced side effects, such as weight gain, depression, or other mood disorders, as the result of the use of the antipsychotic agent, or who are susceptible to such side effects as the result of the use of the antipsychotic agent, are identified. Each individual is instructed to take one 25 mg tablet of topiramate or zonisamide on a daily basis along with 200 mg of bupropion, in addition to the antipsychotic agent therapy.

The individuals are monitored for a period of months, with measurement of symptoms indicative of the efficacy of treatment of the underlying psychotic disorder and relevant side effects. The dosages are adjusted to minimize symptoms of the psychotic disorder and adverse side effects. In the case of weight gain, dosages are typically adjusted so that the patient loses weight at a rate of 10% of initial weight every 6 months. However, the rate of weight loss for each individual can be adjusted by the treating physician based on the individual's particular needs.

The dosage of topiramate can be from about 25 mg to about 1600 mg, preferably from about 50 mg to about 600 mg, more preferably from about 100 mg to about 400 mg. Risperidone is given in daily dosages of between about 0.1 mg and 10 mg, preferably between 1 mg and 5 mg, generally given once per day or divided (e.g., equally) into multiple doses. Risperidone is generally available in 0.25 mg, 0.5 mg, 1 mg, 2 mg, 3 mg, and 4 mg oral dosage units. Olanzapine is given in daily dosages of between about 5 mg and 30 mg, preferably between 5 mg and 15 mg, generally given once per day or divided (e.g., equally) into multiple doses. Olanzapine is typically available in doses of 2.5 mg, 5 mg, 10 mg, 15 mg, or 20 mg. The daily dosage of bupropion can be from about 25 mg to 600 mg, preferably from about 50 mg to 450 mg. Individual tablets, or combination of tablets can be used to achieve the desired dosing. In some instances, it may be necessary to use dosages outside these ranges.

### Example 8

### Combination of Zonisamide with Ziprasidone

Individuals taking ziprasidone, or who are about to take ziprasidone, who have experienced side effects, such as weight gain, depression, or other mood disorders, as the result of the use of the antipsychotic agent, or who are susceptible to such side effects as the result of the use of the antipsychotic agent, are identified. Each individual is instructed to take one 25 mg tablet of zonisamide on a daily basis, in addition to the antipsychotic agent therapy.

The individuals are monitored for a period of months, with measurement of symptoms indicative of the efficacy of treatment of the underlying psychotic disorder and relevant side effects. The dosage is adjusted to minimize symptoms of the psychotic disorder and adverse side effects. In the case of weight gain, dosages are typically adjusted so that the patient loses weight at a rate of 10% of initial weight every 6 months. However, the rate of weight loss for each individual can be adjusted by the treating physician based on the individual's particular needs.

The dosage of zonisamide can be from about 25 mg to about 800 mg per day, generally given once per day or divided (e.g., equally) into multiple doses. Preferably, the dose is from about 100 mg to about 600 mg per day, more preferably, the dose is from about 200 mg to about 400 mg per day. Zonisamide tablets are usually made and marketed in 25 mg, 50 mg, and 100 mg doses. Ziprasidone is given in daily dosages of between about 100 and 400 mg per day, generally given once or twice per day. Individual tablets, or combination of tablets can be used to achieve the desired dosing. In some instances, it may be necessary to use dosages outside these ranges.

### Example 9

### Treatment of Obese Individuals

Individuals suffering from a psychotic disorder having a BMI of greater than 25 are identified. Alternatively, patients are identified having a BMI greater than 30. Each individual is treated and monitored as described above using any of the protocols of Examples 5-8, with particular emphasis on the monitoring of weight loss and symptoms associated with weight loss, such as hypertension, hyperglycemia, etc. Dosages are typically adjusted so that the patient loses weight at a rate of 10% of initial weight every 6 months. However, the rate of weight loss for each individual can be adjusted by the treating physician based on the individual's particular needs.

What is claimed is:

1. A pharmaceutical composition for treating a psychotic disorder, comprising a first ingredient and a second ingredient, wherein the first ingredient comprises an antipsychotic selected from the group consisting of ziprasidone, olanzapine, and risperidone, and wherein the second ingredient comprises an anticonvulsant selected from the group consisting of zonisamide and topiramate.

2. The pharmaceutical composition of claim 1, wherein the antipsychotic is ziprasidone.

3. The pharmaceutical composition of claim 1, wherein the antipsychotic is olanzapine.

4. The pharmaceutical composition of claim 1, wherein the antipsychotic is risperidone.

5. The pharmaceutical composition of claim 2, wherein the anticonvulsant is zonisamide.

6. The pharmaceutical composition of claim 2, wherein the anticonvulsant is topiramate.

7. The pharmaceutical composition of claim 3, wherein the anticonvulsant is zonisamide.

8. The pharmaceutical composition of claim 3, wherein the anticonvulsant is topiramate.

9. The pharmaceutical composition of claim 4, wherein the anticonvulsant is zonisamide.

10. The pharmaceutical composition of claim 4, wherein the anticonvulsant is topiramate.

11. The pharmaceutical composition of claim 1, further comprising an antidepressant.

12. The pharmaceutical composition of claim 11, wherein the antidepressant is a selective serotonin reuptake inhibitor.

13. The pharmaceutical composition of claim 11, wherein the antidepressant is a tricyclic antidepressant.

14. The pharmaceutical composition of claim 11, wherein the antidepressant is an MAO inhibitor.

15. The pharmaceutical composition of claim 11, wherein the antidepressant is a compound that enhances the activity of at least one of norepinephrine and dopamine.

16. The pharmaceutical composition of claim 1, further comprising a physiologically acceptable carrier, diluent, excipient, or a combination thereof.

17. A method of treating a psychotic disorder comprising administering to a patient in need of treatment effective amounts of a first ingredient and a second ingredient, wherein the first ingredient comprises at least one antipsychotic agent selected from the group consisting of ziprasidone, olanzapine, and risperidone, and the second ingredient comprises at least one anticonvulsant selected from the group consisting of zonisamide and topiramate; provided that olanzapine, zonisamide, valproate and bupropion are not simultaneously administered to the patient; and provided that risperidone, zonisamide and paroxetine are not simultaneously administered to the patient.

18. The method of claim 17, further comprising identifying a patient that is undergoing ongoing treatment with at least one antipsychotic selected from the group consisting of ziprasidone, olanzapine, and risperidone.

19. The method of claim 17, further comprising identifying a patient that is suffering from a psychotic disorder associated with one or more symptoms in need of treatment.

20. The method of claim 17, further comprising identifying a patient that is suffering from a psychotic disorder that is in need of mood stabilization.

21. The method of claim 17, wherein said psychotic disorder is selected from the group consisting of bipolar disorders, schizophrenia, borderline personality, schizoid/schizotypical/paranoid personality disorders, delusional disorder, belief reactive psychosis, schizoaffective disorder, schizophreniform disorder, psychotic major depression, psychosis due to substance abuse, psychosis associated with disorders of development, and a psychosis associated with medical conditions.

22. The method of claim 21, wherein said psychosis associated with medical conditions is selected from the group consisting of dementia, delirium, and mental retardation.

23. The method of claim 17, further comprising substantially simultaneously administering the first ingredient and the second ingredient to the patient.

24. The method of claim 17, further comprising administering a pharmaceutical composition to the patient, wherein the pharmaceutical composition comprises the first ingredient and the second ingredient.
